(19) United States
(12) Patent Application Publication
(10) Pub. No.: US 2009/0252802 A1
(43) Pub. Date: Oct. 8, 2009

(54) Compositions Including Relatively Water Insoluble/Unwettable Drugs And Methods For Using Same

(76) Inventors: James N. Chang, Newport Beach, CA (US); Jane-Guo Shiah, Irvine, CA (US)

Correspondence Address: ALLERGAN, INC., 2525 DUPONT DRIVE, T2-7H, IRVINE, CA, 92612-1599, US

(21) Appl. No.: 12/295,882

(22) PCT Filed: Mar. 22, 2007
(86) PCT No.: PCT/US07/64612

§ 371 Date: Feb. 25, 2009

Related U.S. Application Data
(60) Provisional application No. 60/790,332, filed on Apr. 7, 2006.

(51) Int. Cl. A61K 9/14; A61P 43/00

(52) U.S. Cl. 424/489

(57) ABSTRACT

Compositions including therapeutic components and surfactant components which enhance the utility of the therapeutic components and methods of using such compositions are provided.

### CROSS REFERENCE TO RELATED APPLICATIONS

This application is based on, and claims the benefit of, U.S. Provisional Application No. 60/790,332 filed Apr. 7, 2006, and which is incorporated herein by reference.

### BACKGROUND OF THE INVENTION

The present invention relates to compositions including drugs having limited water solubility and limited surface wettability in water, and methods of using such compositions. More particularly, the invention relates to compositions including such drugs, and components which increase or enhance the usefulness or availability of such drugs, and to methods of using such compositions to obtain desired therapeutic effects.

Various drugs, that is pharmaceutically active or therapeutic components, have limited water solubility. Such components are thus limited in usefulness, both from a compounding or dosage form standpoint and from an ultimate utility standpoint. For example, certain alpha-2-adrenergic receptor agonists have limited water solubility. Such limited solubility impairs the ability of such drugs to be absorbed in the gastro-intestinal tracts of humans and animals. In many cases, drugs in the gastro-intestinal tract must be absorbed in the tract in order to be therapeutically effective. The impaired absorption ability of such limited solubility drugs is even further magnified when larger doses of the drugs are required or are administered. In addition, since the drugs have low solubility, they are often limited to be included in a liquid carrier or other form which does not require dissolution, for example, in the gastro-intestinal tract.

One approach to mitigating the limited water solubility of drugs that has been considered is to reduce the size of the drug particles, the theory being that the smaller particles, due to their greater total surface area, would be more readily dissolved in an aqueous environment than larger particles.

In any event, the limitations both to the use of such relatively insoluble and to the dosage forms in which such drugs can be employed present substantial problems or issues as to the usefulness of such drugs even though they are highly effective, for example when absorbed in the gastro-intestinal tract of a human or animal.

There continues to be a need for providing compositions including relatively water insoluble therapeutic components which can be effectively absorbed into the gastro-intestinal tracts of humans or animals.

### SUMMARY OF THE INVENTION

Compositions including therapeutic components which enhance the utility of such therapeutic components and methods for using such compositions have been discovered. The present inventors have found that certain therapeutic components have limited water solubility and limited surface wettability in water, and that both of these properties have an adverse effect on the absorption of such therapeutic components in the gastro-intestinal tracts of humans or animals.

The present compositions effectively enhance the ability of relatively water insoluble and relatively poorly surface wettable therapeutic components to be used in solid forms for effective absorption into a gastro-intestinal tract of a human or animal. In effect, the present compositions address many of the problems with relatively insoluble therapeutic components discussed elsewhere herein, as well as taking into account the relatively poor surface wettability of certain poorly soluble therapeutic components. Solid dosage forms of poorly water soluble and poorly surface wettable therapeutic components in accordance with the present invention provide acceptable and even highly advantageous absorption of such therapeutic components into gastro-intestinal tracts of humans or animals.

The present compositions are straightforward, and easy and cost effective to manufacture. The present methods for using such compositions are easy to practice and effectively provide very useful therapeutic benefits.

In one broad aspect, the present invention is directed to compositions for administration to a gastro-intestinal tract of a human or animal. Such compositions comprise a therapeutic component, a surfactant component and a pharmaceutically acceptable carrier. The therapeutic component, which has limited water solubility and limited surface wettability in water, is present, as a plurality of solid particles, in an amount effective when absorbed in a gastro-intestinal tract of a human or animal to have a desired therapeutic effect on the human or animal. The surfactant component is present in an amount effective to increase the absorption, for example, the amount of absorption and/or the rate of absorption, of the therapeutic component in the gastro-intestinal tract of a human or animal relative to a substantially identical composition without the surfactant component. The surfactant component may be considered to be an absorption enhancer component for the therapeutic component in that the surfactant increases or enhances the amount of or total absorption and/or the rate of absorption of the therapeutic component of the present compositions in the gastro-intestinal tract.

The present compositions are particularly useful with therapeutic components having limited water solubility. For example, and without limitation, the therapeutic component may have a water solubility at 25° C. of less than about 0.5 mg/ml. The present therapeutic components have limited or poor surface wettability in water. For example, such poor surface wettability in water may be attributed to a therapeutic component which is hydrophobic in character. In one embodiment, the presently useful therapeutic component has sufficiently limited surface wettability in water so that solid particles of the therapeutic component are dissolved in water at a reduced rate relative to larger solid particles of the therapeutic component. The presently useful therapeutic component may have contact angles, for example, advancing contact angles, receding or reclining contact angles and the like, such as those contact angles measured by conventional and well known techniques. Such contact angle or angles are related and, therefore, may at least assist in quantifying the limited extent of surface wettability in water of the presently useful therapeutic component. In one embodiment, the present therapeutic components have advancing contact angles of about 90° or greater; and receding contact angles of about 45° or greater. Such contact angles may be measured using the test procedure in which a sessile drop of water or buffered liquid aqueous medium is placed on a tilted surface of the substance being tested.

In one embodiment, the therapeutic component is present as a plurality of solid particles, for example having maximum transverse dimensions or diameters of about 150 microns or less, preferably having maximum transverse dimensions or diameters of about 100 microns or less, for example diameters in a range of about 1 micron to about 30 microns. It has been found that using particles with relatively reduced sizes, as described herein, advantageously enhances the rate of dissolution of the therapeutic component in water and the absorption of the therapeutic component in the gastro-intestinal tract in accordance with the present invention. This is particularly surprising in view of the finding that the present limited surface wettable therapeutic components often have reduced rates of dissolution in water as the particle size of such therapeutic component is reduced.

Any therapeutic component having limited water solubility and poor surface wettability in water as described herein, may be employed in accordance with the present invention. For example, any therapeutic component having limited water solubility, such as set forth herein, and poor surface wettability in water, such as set forth herein, is included within the scope of the present invention.

Of particular note are therapeutic components selected from alpha-2-adrenergic receptor agonist components and mixtures thereof. In one embodiment, alpha-2-adrenergic agonist components lacking sedative activity are employed as therapeutic components in the present invention. Specific examples of alpha-2-adrenergic receptor agonist components and mixtures thereof which are highly useful in accordance with the present invention are described elsewhere herein.

The surfactant component may be selected from any suitable surfactant component effective to function as described herein in accordance with the present invention. In one embodiment, the surfactant component has useful surfactant properties, particularly in the gastro-intestinal tract of a human or animal. The surfactant component is advantageously effective to increase the surface wettability of the therapeutic component in water. Advantageously, the surfactant component is effective both as a surfactant component and as a wettability component. In other words, the present surfactant component advantageously has both surfactant properties and wettability properties.

Without wishing to limit the invention to any particular theory of operation, it is believed that surfactant components having surfactant properties together with an ability to enhance surface wettability of the therapeutic component in water are very effective in enhancing the amount of absorption and/or the rate of absorption of the therapeutic component in the gastro-intestinal tract of a human or animal.

Examples, without limitation, of useful surfactant components include alkali metal hydrocarbyl sulfates, fatty acid ethoxylates, polyoxyethylene stearates, polyoxyethylene/polyoxypropylene copolymers, hypromellose, benzethonium chloride and the like and mixtures thereof.

One very useful surfactant component in the present invention comprises sodium dodecyl sulfate.

The pharmaceutically acceptable carrier may include any suitable excipient or combination of excipients acceptable for use in pharmaceuticals. In one particularly useful embodiment, the carrier is a solid which is highly suitable for use in the gastro-intestinal tract of a human or animal. For example, the carrier component advantageously may dissolve, for example, at least partially dissolve, in such gastro-intestinal tracts.

In one useful embodiment, the present compositions are substantially totally solid at the time the composition is administered to the human or animal. Dosage forms can include, for example and without limitation, tablets, pills, powders, capsules, and the like. Other dosage forms may be used as desired to address the needs of the particular application involved. All such dosage forms can be produced using conventional pharmaceutical manufacturing processes and techniques.

In one aspect of the present invention, methods of treating a human or animal are provided. Such methods comprise orally administering to a human or animal a composition in accordance with the present invention, thereby providing a desired therapeutic effect to the human or animal.

Any feature or combination of features described herein are included within the scope of the present invention provided that the features included in any such combination are not mutually inconsistent as will be apparent from the context, this specification, and the knowledge of one of ordinary skill in the art. In addition, any feature or combination of features may be specifically excluded from any embodiment of the present invention.

These and other aspects and advantages of the present invention are apparent in the following detailed description, drawings, examples and claims.

### BRIEF DESCRIPTION OF THE DRAWINGS

FIG. 1 is a graph showing the dissolution of an alpha-2-adrenergic agonist in a buffer over a time period of 240 minutes.

FIG. 2 is a graph showing the dissolution of an increased amount of the same alpha-2-adrenergic agonist as in FIG. 1 in buffer with time over 120 minutes.

### DETAILED DESCRIPTION OF THE INVENTION

The present compositions and methods provide substantial advantages, for example, in increasing or enhancing the usefulness of drugs which are relatively insoluble and unwettable in water. For example, in accordance with the present invention, such relatively insoluble and unwettable drugs exhibit increased amounts of absorption and/or rates of absorption in the gastro-intestinal tract of a human or animal to which the drug is administered.

In one broad aspect of the present invention, compositions for administration to a gastro-intestinal tract of a human or animal are provided. Such compositions comprise a therapeutic component, a surfactant component, and a pharmaceutically acceptable carrier.

The therapeutic components are present in the present compositions in amounts effective when absorbed in a gastro-intestinal tract of a human or animal to have a desired therapeutic effect on the human or animal. The surfactant component is present in an amount effective to increase the amount of absorption and/or the rate of absorption of the therapeutic component in a gastro-intestinal tract of a human or animal relative to a substantially identical composition without the surfactant component. The pharmaceutically acceptable carrier may be any suitable such carrier including one or more excipients, for example, including one or more conventional and/or well known pharmaceutically acceptable excipients. In one very useful embodiment, the compositions in accordance with the present invention prior to being administered to a gastro-intestinal tract of a human or animal are substantially solid.

Therapeutic components which are of particular usefulness in accordance with the present invention have limited water solubility and limited surface wettability in water, and are at least partially solid prior to being administered to a gastro-intestinal tract of a human or animal. Such therapeutic components may be considered to be hydrophobic. In one embodiment, the therapeutic component has a water solubility at 25° C. of less than about 0.5 mg/ml.

In one embodiment, the therapeutic component has sufficiently limited surface wettability in water so that solid particles of the therapeutic component are dissolved in water, for example, at room temperature, at a reduced rate, for example, for the first 30 minutes or the first 60 minutes after the particles of therapeutic component are placed in the water, relative to larger solid particles of the therapeutic component. For example, and without limitation, the solid particles may have maximum transverse dimensions or diameters of about 100 to about 150 microns (larger particles) and of about 5 microns to about 20 microns (smaller particles). Without wishing to limit the invention to any particular theory of operation, it is believed that therapeutic components which have this property, that is which dissolve in water more slowly as small particles than as larger particles, have sufficiently limited surface wettability in water so as to benefit from inclusion in the present compositions, that is so as to achieve enhanced absorption of the therapeutic component in the gastro-intestinal tract of a human or animal, in accordance with the present invention.

One useful approach to at least assist in quantifying the limited degree or extent of the surface wettability of a therapeutic component in water is to consider, for example, compile or measure, one or more contact angles of the therapeutic component. Contact angles, such as advancing contact angles and receding or reclining contact angles, can be measured by conventional and well known techniques. One or more of such contact angles are often used to determine, quantify or as an indication of the surface wettability of a given substance. In the present instance, the contact angle or angles of the therapeutic component may be considered in determining whether the therapeutic component can be effectively used in the present invention. In one embodiment, the present therapeutic components have advancing contact angles of about 90° or greater; and receding contact angles of about 45° or greater. Such contact angles may be measured using the test procedure in which a sessile drop of water or buffered liquid aqueous medium is placed on a tilted surface of the substance being tested. This test procedure is illustrated in the Examples herein.

A therapeutic component is considered relatively less wettable by the liquid, e.g. water if, the measured advancing and receding contact angles are relatively large. In other words, the less surface wettable a therapeutic component is the greater the advancing and receding contact angles of the therapeutic component are. For example, a therapeutic component in accordance with the present invention may have an advancing contact angle of about 90° or greater, such as about 100° or greater, or about 110° or greater. A therapeutic component in accordance with the present invention may have a receding contact angle of about 45° or greater, such as about 50° or greater or about 55° or greater or about 60° or greater.

The present compositions may include therapeutic components present in any solid form. A very useful form for the therapeutic component to take is that of a plurality of particles. The plurality of particles may be free flowing, located in a capsule, present in a pill tablet, powder, and the like solid forms. Without wishing to limit the invention to any particular theory of operation, it is believed that having the therapeutic component present as a plurality of particles provides an increased surface area which allows the present surfactant component to interact with the increased surface of such particles to reduce surface tension, enhance surface wettability and ultimately to increase the solubility or apparent solubility of the therapeutic component in the fluids in the gastro-intestinal tract, making the therapeutic component more available (in a solubilized form) for absorption into the gastro-intestinal tract relative to the solid form of the therapeutic component without the surfactant.

In one useful embodiment, the therapeutic component is present in a plurality of particles having a maximum transverse dimensions or diameters of less than about 150 microns, for example, about 100 microns or less. Advantageously, a major amount, that is at least about 50%, by weight of the particles have maximum transverse dimensions or diameters in a range of about 1 micron to about 30 microns or about 50 microns. In one embodiment, at least about 70% or at least about 80% or at least about 90% by weight of the particles have such sizes. Very useful results are achieved when substantially all of the particles have such sizes.

In one very useful embodiment, a major amount, that is at least about 50%, by weight of the particles have diameters of about 1 micron or about 3 microns to about 10 microns or about 20 microns or about 30 microns. In one embodiment, at least about 70% or at least about 80% or at least about 90% by weight of the particles have such sizes. Very useful results are achieved when substantially all of the particles have such sizes.

The therapeutic component having particle sizes as described herein can be obtained using conventional techniques and equipment well known in the pharmaceutical art. For example, the therapeutic component, in the form of a dry mixture of relatively large particles, can be micronized to particles having the desired size, for example, about 10-20 microns, using a mill, such as an Aljet mill (Model 00 Jet-0-Mizer, Fluid Energy, Telfors, Pa.). If necessary, the resulting mixture of therapeutic component particles can be screened or otherwise separated to obtain therapeutic component particles of the desired size or size range.

Any suitable therapeutic component may be used in accordance with the present invention. Advantageous results are achieved with therapeutic components which have limited water solubility and limited surface wettability in water, for example as described elsewhere herein.

Among the therapeutic components which may be employed in the present compositions include, without limitation, those substances, for example, compounds and mixtures of such substances, which are useful to provide a therapeutic benefit or effect when administered to a human or animal. The therapeutic components useful in this invention include, without limitation, NMDA antagonists, antibacterials, antihistamines, decongestants, antiinflammatories, antiparasitics, miotics, anticholinergics, adrenergics, antivirals, local anesthetics, antifungals, amoebicidals, trichomonocidals, analgesics, mydriatics, antiglaucoma drugs, neuroprotective agents, antiaugiogenic agents, chelating agents, antineoplastics, antihypertensives, muscle relaxants, diagnostics and the like and mixtures thereof.

In one very useful embodiment, the therapeutic component is selected from alpha-2-adrenergic receptor agonists components and mixtures thereof. Such agonist components provide substantial benefits when administered to gastro-intestinal tracts of humans or animals, and often have limited solubility in water and limited surface wettability in water.

One particularly useful class of therapeutic components useful in accordance with the present invention are alpha-2-adrenergic receptor agonist components lacking sedative activity. Examples of such components, methods of their making, and methods of screening such components are provided, for example and without limitation, in the following patents and patent publications: U.S. Pat. Nos. 6,329,369; 6,545,182; 6,841,684 and U.S. Patent Publications Serial No. US20020161051, entitled “(2-hydroxy)ethyl-thioureas Useful as Modulators of Alpha2B adrenergic receptors”; US20030023098, entitled “Compounds and Method of Treatment Having Agonist-like Activity Selective at Alpha 2B or 2B/2C Adrenergic Receptors”; US20030092766, entitled “Methods and Compositions for Modulating Alpha Adrenergic Receptor Activity”; US20040220402, entitled “4-(substituted cycloalkylmethyl) imidazole-2-thiones, 4-(substituted cycloalkenylmethyl) imidazole-2-thiones, 4-(substituted cycloalkylmethyl) imidazol-2-ones and 4-(substituted cyloalkenylmethyl) imidazol-2-ones and Related Compounds”; US20040266776, entitled “Methods of Preventing and Reducing the Severity of Stress-associated Conditions”; US20050059664 entitled “Novel Methods for Identifying Improved, Non-sedating alpha-2 agonists”; US20050059721, entitled “Nonsedating Alpha-2 Agonists”; US20050059744 entitled “Methods and Compositions for the Treatment of Pain and Other Alpha 2 adrenergic-mediated Conditions”; and US20050075366 entitled “4-(2-Methyl-5,6,7,8-tetrahydroquinoline-7-ylmethyl)-1,3-dihydro-imidazole-2-thione as Specific alpha2B Agonist and Methods of Using the Same”. Additional disclosure concerning non-sedating alpha 2 adrenergic agonists can be found in US20050058696, entitled Methods and Compositions for the Treatment of Pain and Other Alpha 1 Adrenergic Mediated Conditions”, and US20040132824, entitled “Novel Methods and Compositions of Alleviating Pain”. All the patents and patent publications (applications) referenced herein are incorporated by reference herein in their entireties.

Particularly useful alpha-2-adrenergic receptor agonist components useful in accordance with the present invention are selected from:

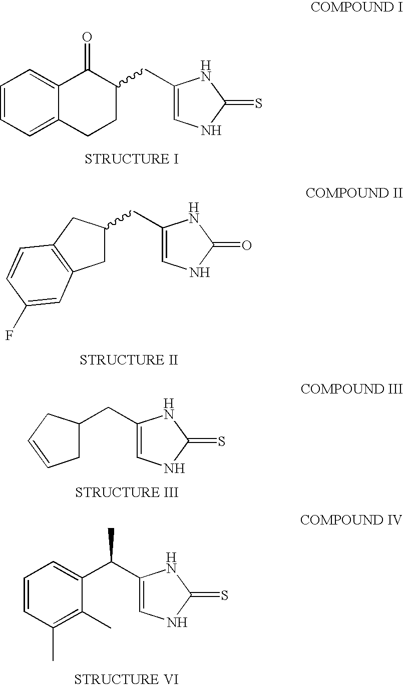

pharmaceutically acceptable salts, esters, amides, stereoisomers, and racemic mixtures thereof, and mixtures thereof. Each of these components is identified in one or more of the patents/patent publications which are incorporated in their entireties by reference herein. Pharmaceutically acceptable salts, esters, amides, stereoisomers or racemic mixtures of Compounds I-IV can be prepared by routine methods. The agonists of Compounds I-IV are merely exemplary of a variety of salts, esters, amides, etc. of these Compounds that can be readily prepared by one skilled in the art using well known methods of chemical synthesis.

Any suitable surfactant component effective to facilitate or increase the absorption of the therapeutic component in the gastro-intestinal tract of a human or animal as described elsewhere herein may be employed in accordance with the present invention.

The presently useful surfactant components advantageously have surfactant properties in the gastro-intestinal tract of a human or animal, for example, a mammalian animal. The surfactant components may be effective to increase the surface wettability of the therapeutic component in water, for example, in the gastro-intestinal tract of a human or animal. In one useful embodiment, the surfactant component is effective as a surfactant, that is has surfactant properties, and as a wettability or wetting agent, that is has wettability or wetting properties, in accordance with the present invention.

Among the useful surfactant components are included, without limitation, surfactants selected from alkali metal hydrocarbyl sulfates, fatty acid ethoxylates, for example, polyoxyethylene sorbitan fatty acid ester ethoxylates, such as polysorbate 20-80 and the like, polyoxyethylene stearates, polyoxyethylene/polyoxypropylene copolymers, for example, block copolymers, such as those sold under the trademark Pluronic® and Tetronic®, hypromellose and the like and mixtures thereof.

A particularly useful class of surfactant components for the present invention are those selected from alkali metal hydrocarbyl sulfates having about 8 to about 16 carbon atoms per molecule and mixtures thereof. Sodium hydrocarbyl sulfates having about 8 to about 16 carbon atoms per molecule and mixtures thereof are very useful. Alkali metal, for example, sodium, alkyl sulfates having 8 to about 16 carbon atoms per molecule and mixtures thereof are particularly useful. Excellent results are obtained when the surfactant component comprises sodium dodecyl sulfate, which is also known as sodium lauryl sulfate.

The surfactant component is present in the compositions in an amount effective to increase the absorption, that is the amount of absorption and/or the rate of absorption, of the therapeutic component in a gastro-intestinal tract of a human or animal. The amount of any particular surfactant component included in the present composition may vary over a relatively wide range depending, for example and without limitation, on the therapeutic component used, on the specific surfactant component used, and on the amount and/or rate of therapeutic component absorption desired, among other factors. In one embodiment, the surfactant component may be present in the present composition in an amount in a range of about 0.01% (wt) or less to about 10% (wt) or about 15% (wt) or more, preferably in a range of about 0.2% (wt) or about 0.5% (wt) to about 4% (wt) or about 6% (wt) of the total composition.

The compositions of the present invention further include pharmaceutically acceptable carriers, which is any carrier, excipient or diluent that has substantially no long term or permanent detrimental effect when administered to a subject, that is a human or animal. An excipient generally is mixed with an active therapeutic component, or permitted to dilute or enclose the therapeutic component. A carrier can be a solid or semi-solid agent that acts as an excipient or vehicle for the therapeutic component. Examples of solid carriers useful in the pharmaceutical compositions of the invention include, without limitation, pharmaceutical grades of mannitol, lactose, starch, magnesium stearate, sodium saccharin, polyalkylene glycols, talcum, cellulose, glucose, sucrose and magnesium carbonate.

Examples of such materials which are very useful in the present compositions include, without limitation, filler components or diluent components, disintegrant components, glidant components, lubricant components, buffer components, preservative components, capsule components and the like and mixtures thereof. As noted above, the material or materials making up the pharmaceutically acceptable carrier may be chosen from conventional materials which are well known in the pharmaceutical art. Any suitable material or materials may be used in the pharmaceutically acceptable carrier provided that such material or materials are pharmaceutically acceptable in the application in which the present composition is to be used, do not unduly interfere with the pharmaceutical activity of the therapeutic component included in the present composition, do not unduly interfere with the surfactant component in the present composition, and function as intended in the present composition. The amount of each material included in the pharmaceutically acceptable carrier may be chosen to perform one or more desired functions.

Examples of useful filler components/diluent components include, without limitation, calcium carbonate, calcium phosphate; cellulose, such as (without limitation) microcrystalline cellulose, other forms of cellulose and the like; cellulosic derivatives, such as (without limitation) cellulose acetate, ethylcellulose and the like; dextrin and the like; sugars, such as (without limitation) dextrose, lactose, maltose, sucrose and the like; glyceryl palmitostearate and the like; mannitol, sorbitol, xylitol and the like; polymethacrylates and the like; sodium alginate and the like; starch, starch derivatives and the like; talc and the like; and the like and mixtures thereof. The filler component/diluent component may be present in a widely varying amount in the present compositions. The filler component/diluent component may comprise at least about 30% (wt), for example, up to about 90% (wt) of the composition and may be increased or decreased depending on the presence and amounts of other materials in the composition.

The disintegrant component may be selected from the following exemplary materials including, without limitation, alginic acid, sodium alginate and the like; tribasic calcium phosphate and the like; cellulose, such as (without limitation) microcrystalline cellulose, other forms of cellulose and the like; cellulose derivatives, such as (without limitation) sodium carboxymethylcellulose, hydroxypropyl cellulose, methylcellulose and the like; chitosan and the like; silicon-containing materials, such as (without limitation) colloidal silicone dioxide, magnesium aluminum silicate and the like; croscarmellose sodium and the like; crospovidone and the like; povidone and the like; starch and starch derivatives, such as (without limitation) sodium starch glycolate, and the like; and the like and mixtures thereof. The disintegrant component may be present in the present composition in an amount effective to assist or facilitate the break up or disintegration of the dosage form, for example, pill, capsule and the like, of the composition in the gastro-intestinal tract of a human or animal, for example, as a preliminary step in releasing the therapeutic component from the dosage form for absorption into the gastro-intestinal tract.

As can be seen from the examples of filler/diluent components and disintegrant components noted above, some overlap of these components exists. Thus, in certain instances, the disintegrant component may be part of the filler/diluent component and vice versa.

The disintegrant component advantageously is present in the present compositions in an amount in a range of about 1% (wt) or about 5% (wt) to about 20% (wt).

The combination of the filler/diluent component and the disintegrant component is advantageously effective to deliver the dosage form of the present composition to the gastro-intestinal tract of the human or animal and to facilitate or assist in releasing the therapeutic component from the dosage form.

The glidant component may be selected from the following examples, without limitation, tribasic calcium phosphate and the like; cellulose and cellulosic derivatives, for example, (and without limitation) as described elsewhere herein, and the like; silicon-containing materials, such as (without limitation) colloidal silicon dioxide, calcium silicate, magnesium silicate and the like; starch, starch derivatives and the like; talc and the like; and the like and mixtures thereof.

The glidant component may be present in an amount effective to facilitate the passage of the orally administered dosage form of the composition to the gastro-intestinal tract. For example, the glidant component may be present in the compositions in an amount in a range of about 0.1% (wt) or less or about 0.3% (wt) to about 1.5% (wt) or about 3% (wt) or more.

The lubricant component may be present in an amount effective to facilitate the formation of the dosage forms of the present composition.

Examples of useful lubricant components include, without limitation, stearic acid and stearates, such as (without limitation) calcium stearate, magnesium stearate, glycerine monostearate, and the like; polyethylene glycols and the like; polyoxyethylene-polyoxypropylene copolymers (polaxamer); and the like and mixtures thereof.

Such lubricant component may be present in the present compositions in an amount in a range of about 0.1% (wt) or less to about 1.0% (wt) or more.

The present compositions may include one or more agents such as, without limitation, emulsifying agents, sweetening or flavoring agents, tonicity adjusters, preservatives, buffers, antioxidants and the like. Tonicity adjustors useful in a pharmaceutical composition of the invention include, but are not limited to, salts such as sodium acetate, sodium chloride, potassium chloride, mannitol or glycerin and other pharmaceutically acceptable tonicity adjustors. Preservatives useful in the pharmaceutical compositions of the invention include, without limitation, benzalkonium chloride, chlorobutanol, thimerosal, phenylmercuric acetate, and phenylmercuric nitrate. Various buffers and means for adjusting pH can be used to prepare a pharmaceutical composition, including, but not limited to, acetate buffers, citrate buffers, phosphate buffers and borate buffers. Similarly, anti-oxidants useful in pharmaceutical compositions are well known in the art and include, for example, sodium metabisulfite, sodium thiosulfate, acetylcysteine, butylated hydroxyanisole and butylated hydroxytoluene. It is understood that these and other substances known in the art of pharmacology can be included in a pharmaceutical composition of the invention. See, for example, Remington's Pharmaceutical Sciences Mack Publishing Company.

The therapeutic components included in the present compositions may be effective in providing any one or more therapeutic effects to the human or animal to whom the compositions are administered. Advantageously, the compositions are administered to the human or animal so that the therapeutic component is absorbed in the gastro-intestinal tract of the human or animal. For example, the composition may be orally administered to the human or animal.

In one embodiment, for example and without limitation, the therapeutic component is an alpha 2-adrenergic receptor agonist component, such as one which lacks sedative activity, and can be useful, for example and without limitation, in preventing or reducing (alleviating) a sympathetically-enhanced condition, such as without concomitant sedation.

Any of a variety of sympathetically-enhanced conditions can be prevented or alleviated without concomitant sedation by a therapeutic component in accordance with the present invention, including, without limitation, sensory hypersensitivity such as that associated with fibromyalgia or headaches such as migraines; gastro-intestinal diseases such as irritable bowel syndrome and dyspepsia; dermatological conditions such as psoriasis; cardiovascular disorders; tachycardias; disorders of peripheral vasoconstriction including Raynaud's Syndrome and scleroderma; panic attacks; metabolic disorders such as type II diabetes, insulin-resistance and obesity; disorders of muscle contraction including disorders of skeletal muscle contraction, disorders of smooth muscle contraction, spasticity, and disorders of muscle contraction associated with tension-type headache; behavioral disorders such as, but not limited to, over-eating and drug dependence; and sexual dysfunction.

The therapeutic component provided herein also can be useful, for example, for prevention or alleviation of chronic pain. Chronic pain is a term which means pain other than acute pain and includes, without limitation, neuropathic pain, visceral pain, inflammatory pain, headache pain, muscle pain and referred pain. It is understood that chronic pain is of relatively long duration, for example, several years and can be continuous or intermittent. Chronic pain is distinguished from acute pain, which is immediate, generally high threshold, pain brought about by injury such as a cut, crush, burn, or by chemical stimulation such as that experienced upon exposure to capsaicin, the active ingredient in chili peppers.

Any of a variety of types of chronic pain can be prevented or alleviated without concomitant sedation by an alpha 2 adrenergic receptor agonist component in accordance with the invention including, but not limited to, neuropathic pain such as neuropathic pain associated with diabetic neuropathy or post-herpetic neuralgia; chronic pain associated with cancer; post-operative pain; allodynic pain such as fibromyalgic pain; chronic pain associated with Complex Regional Pain Syndrome (CRPS); chronic visceral pain such as that associated with irritable bowel syndrome or dysmennorhea; chronic headache pain such as migraine pain, non-vascular headache pain, cluster headache pain or daily tension headache pain; and chronic muscle pain such as, yet not limited to, that associated with back spasm.

The therapeutic component provided herein additionally can be useful, for example, in preventing or alleviating a neurological condition. Such a neurological condition can be, without limitation, an acute or chronic neurological condition. As non-limiting examples, acute neurological conditions which can be prevented or alleviated include stroke; head and spinal cord trauma; and seizure. Furthermore, chronic neurological conditions which can be prevented or alleviated include, but are not limited to, neurodegenerative diseases such as Alzheimer's disease; Parkinson's disease; Huntington's disease; amyotrophic lateral sclerosis and multiple sclerosis; HIV-associated dementia and neuropathy; ocular diseases such as glaucoma, diabetic neuropathy and age-related macular degeneration; and schizophrenia, drug addiction, withdrawal and dependency, and depression and anxiety.

The term neurological condition encompasses all acute and chronic disorders which affect, at least in part, neurons. Thus, the term neurological condition encompasses, without limitation, hypoxia-ischemia (stroke); head and spinal cord injury; epilepsy; neurodegenerative disorders such as Alzheimer's disease, Parkinson's disease, Parkinsonism; Huntington's disease, amyotrophic lateral sclerosis and multiple sclerosis; optic neuropathies such as glaucoma, light-induced retinal degeneration such as photoreceptor degeneration, and macular degeneration; disorders of photoreceptor degeneration such as retinitis pigmentosa; HIV-associated dementia (acquired immunodeficiency syndrome dementia complex) and HIV-associated neuropathy; metabolic, mitochondrial and infectious brain abnormalities such as, but not limited to, encephalitis; neuropathic pain syndromes such as causalgia or painful peripheral neuropathies; olivopontocerebellar atrophy; mitochondrial abnormalities and other biochemical disorders such as MELAS syndrome, MERRF, Leber's disease, Wernicke's encephalopathy, Rett syndrome, homocysteinuria, hyperhomocysteinemia, hyperprolinemia, nonketotic hyperglycinemia, hydroxybutyric aminoaciduria, sulfite oxidase deficiency, combined systems disease, lead encephalopathy; hepatic encephalopathy, Tourette's syndrome; drug addiction and drug dependency; drug withdrawal such as withdrawal from alcohol or opiates; and depression or anxiety syndromes (see, for example, Lipton and Rosenberg, New Enql. J. Med. 330: 613 (1994)).

Any of a variety of ocular conditions can be prevented or alleviated without concomitant sedation following peripheral administration of an alpha-2-adrenergic receptor agonist component in accordance with the present invention. Such conditions include, without limitation, diabetic retinopathy; macular edema such as that associated with diabetes; conditions of retinal degeneration such as glaucoma, macular degeneration such as age-related macular degeneration (ARMD) and retinitis pigmentosa; retinal dystrophies; inflammatory disorders of the retina; vascular occlusive conditions of the retina such as retinal vein occlusions or branch or central retinal artery occlusions; retinopathy of prematurity; retinopathy associated with blood disorders such as sickle cell anemia; elevated intraocular pressure; ocular itch; damage following retinal detachment; damage or insult due to vitrectomy, retinal or other surgery; and other retinal damage including therapeutic damage such as that resulting from laser treatment of the retina, for example, pan-retinal photocoagulation for diabetic retinopathy or photodynamic therapy of the retina. Ocular conditions that can be prevented or alleviated further include, without limitation, genetic and acquired optic neuropathies such as optic neuropathies characterized primarily by loss of central vision, for example, Leber's hereditary optic neuropathy (LHON), autosomal dominant optic atrophy (Kjer disease) and other optic neuropathies such as those involving mitochondrial defects, aberrant dynamin-related proteins or inappropriate apoptosis; and optic neuritis such as that associated with multiple sclerosis, retinal vein occlusions or photodynamic or laser therapy. See, for example, Carelli et al., Neurochem. Intl. 40:573-584 (2002); and Olichon et al., J. Biol. Chem. 278:7743-7746 (2003). It is understood that these and other ocular abnormalities, especially those of the neurosensory retina, can be prevented or alleviated without concomitant sedation using the selective alpha-2-adrenergic receptor agonist components in accordance with the present invention.

The present therapeutic components can be useful for preventing or alleviating other disorders, for example, without concomitant sedation. Such a disorder can be, for example and without limitation, attention deficit disorder (ADHD/ADD), which is a disorder primarily characterized by inattention, distractibility and impulsiveness starting before the age of seven. Symptoms can include, without limitation, fidgeting and squirming, difficulty in remaining seated, easy distractibility, difficulty awaiting one's turn, difficulty in refraining from blurting out answers, inability to follow instructions, excessive talking, and other disruptive behavior (Anderson, supra, 1994). Furthermore, while originally recognized in children, ADHD/ADD continues into adulthood in many individuals (see, for example, Block, Pediatr. Clin. North Am. 45:1053-1083 (1998); and Pary et al., Ann. Clin. Psychiatry 14:105-111 (2002)). One skilled in the art understands that a method of the invention can be useful for preventing or alleviating ADHD/ADD in children and adults having mild as well as severe forms of the disorder.

The present therapeutic components also can be useful to prevent or alleviate nasal congestion; diarrhea; urinary disorders such as hyperactive micturition and overactive bladder; congestive heart failure; or a psychosis such as a manic disorder. Furthermore, such therapeutic components can be useful to prevent or alleviate one or more symptoms associated with anesthesia such as nausea, vomiting, shivering or panic; or to enhance memory and cognitive processes, for example, without limitation, without concomitant sedation.

The term “alleviating,” as used herein, means reducing by at least about 50% at least one symptom of the particular condition or type of chronic pain being treated.

As is well known in the art, sedation is a term that means a reduction in motor activity. The phrase “without concomitant sedation,” as used herein with reference to a therapeutic component, for example, a selective alpha-2-adrenergic receptor agonist component, means that, upon peripheral administration, for example, oral administration, the component produces less than about 30% sedation at a dose 10-fold greater than the dose of component required to produce a 50% reduction of one or more symptoms of the particular condition or type of chronic pain being treated. In contrast, many alpha-2-adrenergic receptor agonists, such as dexmeditomidine, are completely sedating at doses 10-fold greater than the dose required to produce a 50% reduction in one or more symptoms of the particular condition or type of chronic pain being treated.

As non-limiting examples, the dose of alpha-2-adrenergic receptor agonist component in accordance with the present invention required to produce about 30% sedation (reduction in motor activity) can be at least 25-fold greater than, 50-fold greater than, 100-fold greater than, 250-fold greater than, 500-fold greater than, 1000-fold greater than, 2500-fold greater than, 5000-fold greater than, or 10,000-fold greater than the dose required to produce a 50% reduction in one or more symptoms of the particular condition or type of chronic pain being treated. Methods for determining the extent of a reduction in a symptom as well as the extent of sedation are well known in the art.

The following non-limiting examples illustrate certain aspects of the present invention.

### Example A

The tilting plane method of measuring contact angles involves slowly tilting a surface or plane of a substance the contact angle of which is to be determined until a sessile drop on the plane begins to move in the downhill direction. At that time the angle between the plane and the downhill tangent of the drop at the plane is the advancing contact angle, and the angle between the plane and the uphill tangent of the drop at the plane is the receding contact angle.

A procedure for determining the contact angles of a therapeutic component is as follows.

A small batch of Compound III weighing approximately 30 g is micronized in a ball mill for three days using 5-mm stainless steel balls. The resulting particles have an average diameter of 8 μm. A 200-mg sample of the micronized Compound III is compressed into a wafer 12 mm in diameter and 2 mm thick using a Carver press. The wafer is then glued on to a plastic board.

Separately, a 10 milliliter aqueous solution is prepared containing 50 mM sodium phosphate buffer at pH 6.8. A drop of a blue food dye is added to the solution to aid visualization.

A 40 μL drop of the blue aqueous solution is placed on top of the Compound III wafer on a plastic board resting at a horizontal position. The plastic board and the wafer are then slowly tilted from their horizontal position in front of a continuous shooting camera. The process stops after the blue aqueous drop has run down the wafer. The images that show the shapes of the liquid drop just prior to it starting to run down the wafer are analyzed and the contact angles measured.

For Compound III, it has been determined that the advancing and receding contact angles are 116° and 73°, respectively.

Alternatively, both the advancing and receding contact angles can be measured using a KSV instrument (model CAM 200) with a tilt stage. The instrument is fully computer controlled, and it captures the video image and performs full analysis of the advancing and receding contact angles.

### Examples 1 to 4

Samples A, B, C and D of an alpha-2-adrenergic receptor agonist having the following structure

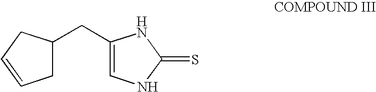

(Compound III) are selected for testing. Compound III have very limited water solubility and surface wettability in water. For example, the solubility of Compound III [AGN 199981] in water at 25° C. is less than 0.5 mg/ml, and the advancing/receding contact angles of Compound III are 116°/73°, respectively.

Samples A and B are in the form of particles having an average diameter of about 150 microns. Samples C and D are in the form of particles having an average diameter of about 10 microns.

An aqueous solution buffered with a buffer to a pH of 6.8 is provided. A quantity of a mixture containing conventional pharmaceutically acceptable excipients is provided. In addition, a quantity of sodium dodecyl sulfate is provided.

A mixture of Sample A (100 mg) and excipients is formed and is placed in 900 ml of the aqueous solution.

A mixture of Sample C (100 mg) and excipients is formed and is placed in another 900 ml of the aqueous solution. Test samples of each of the aqueous solutions are taken at various times after each of the mixtures containing Compound III (Samples A and C) is placed in the aqueous solution. Each of these test samples is analyzed for Compound III concentration. Using these test results over a period of 240 minutes, relationships between the percentage of Sample A and Sample C dissolved and time are developed. These tests are run a total of 3 times (n=3).

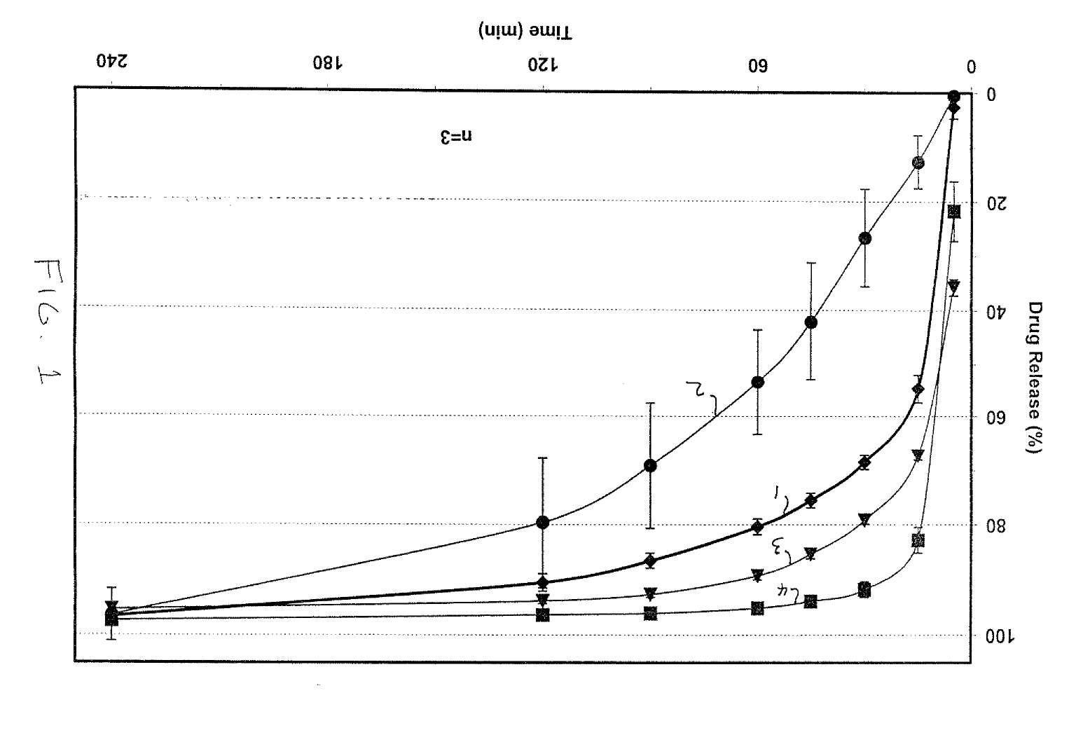

Such relationships are graphically illustrated by the data points through which Curve 1 (Sample A) and Curve 2 (Sample C) in FIG. 1 are drawn.

The relationship between Curve 1 and Curve 2 is quite surprising. These curves demonstrate that larger particles of Compound III (in Sample A, Curve 1) dissolve more quickly in the aqueous buffer solution than do smaller particles of Compound III (in Sample C, Curve 2). One would intuitively expect smaller solid particles to dissolve more quickly than larger solid particles. To this extent at least, the fact that larger particles of Compound III (in Sample A, Curve 1) dissolve more quickly than smaller particles of Compound III (Curve 2) is unexpected. Without wishing to limit the present invention to any particular theory of operation, it is believed that the combination of low water solubility and low surface wettability of Compound III results in a reduced rate of dissolution of the smaller particles of Compound III, for example, because a given quantity (weight) of the smaller particles has an increased surface area than the same quantity (weight) of the larger particles.

In any event, Compound III also has a relatively reduced rate of dissolution in water, whether present as small particles or as large particles. Such reduced dissolution rate can limit the absorption of Compound III, as well as such other therapeutic agents, in the gastro-intestinal tract of a human or animal which, in turn, can result in reduced therapeutic effectiveness.

Each of Samples B and D is tested in a substantially similar way. However, when testing Samples B and C, 1% based on weight of the mixture of Sample and excipient mixture, of sodium dodecyl sulfate is included in the mixture. Each of these tests is run a total of 3 times.

The relationships between Compound III dissolved and time for Samples B and D are graphically illustrated by the data points through which Curves 3 and 4, respectively, in FIG. 1 are drawn.

Comparing Curves 1-4 in FIG. 1 shows that each of the relationships is different. In particular, with regard to Curves 1 and 2, it is apparent that relatively large (150 microns) and relatively small (10 microns) particles of the Compound III without any sodium dodecyl sulfate dissolve relatively slowly, particularly in time periods of about 120 minutes or less.

This “slow” solubility of relatively large Compound III particles is mitigated to some extent by the presence of sodium dodecyl sulfate, which has surfactant properties, as shown by comparing Curves 1 and 3 of FIG. 1. Thus, the use of sodium dodecyl sulfate surfactant, which also has wettability properties, advantageously increases the water solubility of therapeutic agents which have limited water solubility and limited surface wettability, such as Compound III.

In addition, by comparing Curves 2 and 4 of FIG. 1, it is apparent that reducing the size of the Compound III particles provides further solubility increases. Without wishing to limit the invention to any particular theory of operation, it is believed that materials which have surfactant properties and provide increased wettability, such as sodium dodecyl sulfate, are particularly useful in increasing therapeutic component solubility when smaller therapeutic component particles, with increased overall particle surface area, are used.

It is important to note that, in the absence of a surfactant with wettability properties, reducing the size of the Compound III particles actually reduces the rate of dissolution relative to larger particles of Compound III. Compare Curves 1 and 2 of FIG. 1. However, in the presence of a surfactant with wettability properties, reducing the size of the Compound III particles further increases the rate of dissolution relative to larger particles of Compound III. Compare Curves 3 and 4 of FIG. 1. Increases in the rate of dissolution of such therapeutic components are highly advantageous and allow increased absorption of such therapeutic components in the gastro-intestinal tract of a human or animal.

In any event, as shown in FIG. 1, the use of relatively small particles of the above-noted agonist together with a wettability-enhancing surfactant, such as sodium dodecyl sulfate, substantially increases the solubility of the Compound III, which has limited water solubility and limited surface wettability in water. The solubilized Compound III is more readily absorbed in the gastro-intestinal tract and, as such, is more effective to provide the desired therapeutic effect.

### Examples 5 and 6

The tests described in Examples 1 to 4 with Samples A and B are repeated, except that the mixtures used included Sample A (200 mg) and excipients, and Sample B (200 mg) and excipients.

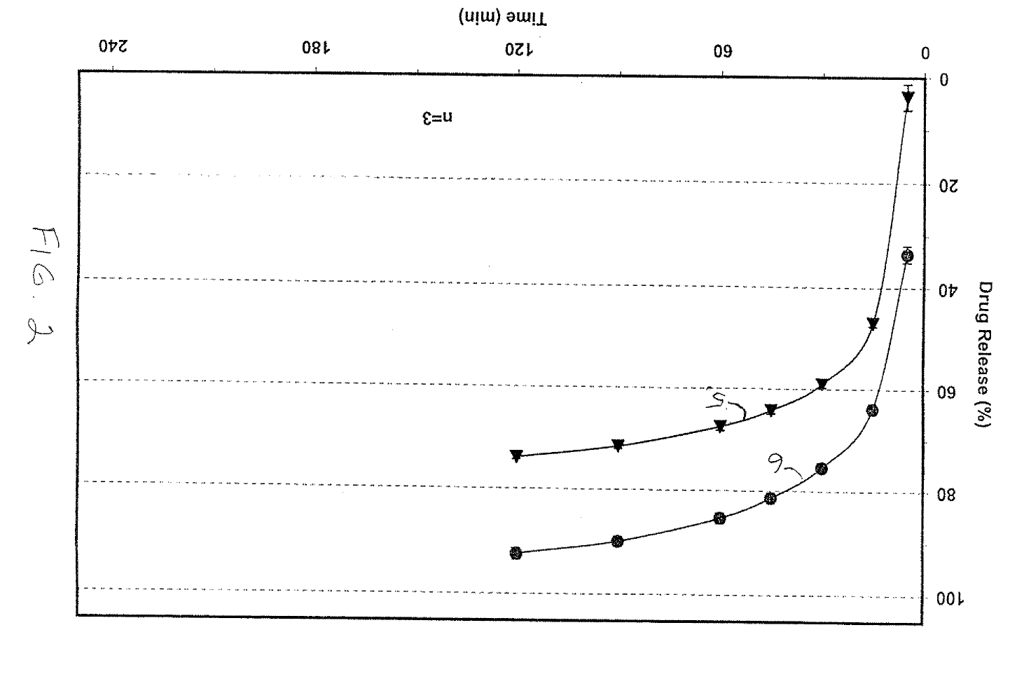

Results of these tests are shown in FIG. 2. Curve 5 represents the relationship obtained of % (percent) drug released or dissolved verses time in contact with the aqueous solution for Sample A. Similarly, Curve 6 represents such relationship obtained for Sample B.

These test results confirm the test results shown in FIG. 1. In particular, comparing Curve 6 with Curve 5, it is apparent that the presence of a wettability-enhancing surfactant, such as sodium dodecyl sulfate, increases the water solubility (or increases the rate of water solubility) of the relatively insoluble agonist.

Moreover, Curve 5 demonstrates that this increased dissolving or solubility effect of such surfactants occurs to provide increased amounts of such therapeutic components in solution (and, therefore, absorbable in the gastro-intestinal tract of a human or animal) even with increased dosages of such therapeutic components.

To illustrate, at 60 minutes in the buffer solution, Curve 1 indicates that 80% of 100 mg or 80 mg of the agonist has been dissolved.

At 60 minutes in the buffer solution, Curve 3 indicates that 88% of 100 mg or 88 mg of the agonist have been dissolved.

At 60 minutes in the buffer solution, Curve 5 indicates that 67% of 200 mg or 134 mg of the agonist has been dissolved.

At 60 minutes in the buffer solution, Curve 6 indicated that 85% of 200 mg or 170 mg of the agonist has been dissolved.

These results make clear that the enhanced solubility (or the enhanced rate of solubility) of relatively insoluble, relatively unwettable therapeutic components obtained with the wettability-enhancing surfactants in accordance with the present invention is substantially beneficial even if increased amounts of the therapeutic component are used or needed to obtain the desired therapeutic effect.

In short, in accordance with the present invention increased amounts of relatively water insoluble, relatively unwettable therapeutic components, such as alpha-2-adrenergic receptor agonist Compound III, are available for absorption in the gastro-intestinal tract of a human or animal to provide desired therapeutic effects even in situations where relatively large doses of such therapeutic components are required or used.

### Examples 7 and 8

A series of two compositions are prepared as follows.

Particles of Compound I, having a chemical structure as defined elsewhere herein, are provided which have diameters in excess of 100 microns. These particles are micronized in an Aljet mill (Model 00 Jet-0-Mizer) to produce particles of Compound I having diameters in a range of 10-20 microns. Compound I has a solubility in water at 25° C. of less than 0.5 mg/ml; and advancing/receding contact angles of greater than 90°/greater than 45°, respectively.

These small particles of Compound I are combined with particles of sodium dodecyl sulfate, a filler/diluent, such as microcrystalline cellulose, a disintegrant, such as pregelatinized starch, a glidant, such as fumed silica, and a lubricant, such as magnesium stearate, to form two different compositions. The combined materials are mixed in blenders. The two mixtures have the following chemical make-ups.

CONCENTRATION, WT %

| | EXAMPLE | EXAMPLE |
|---|---|---|
| INGREDIENT | 7 | 8 |
| Compound I | 25 | 25 |
| Sodium Dodecyl Sulfate | 1.5 | 1.0 |
| Microcrystalline Cellulose | 64.5 | 65 |
| Pregelatinized Starch | 7.5 | 7.5 |
| Fumed Silica | 1.0 | 1.0 |
| Magnesium Stearate | 0.5 | 0.5 |

### Examples 9 and 10

One portion of each of Compositions 7 and 8 is pressed into a tablet, using a conventional tablet press. Each of the tablets includes 100 mg of Compound I, and is useful to be orally administered to a human or animal.

### Examples 11 and 12

Another portion of each of Compositions 7 and 8 is encapsulated into a gelatin-based capsule, using conventional capsule filling techniques. Each of the capsules includes 100 mg of Compound I, and is useful to be orally administered to a human or animal.

### Examples 13 and 14

A further portion of each of Compositions 7 and 8 is combined with a sufficient amount of water and mixed to form an extrudable mixture. Using conventional extrusion, cutting and drying equipment and techniques, each of the extrudable mixtures is extruded into thin strands, which are cut to form beads of suitable size to be placed into a capsule, and dried. After the beads are dried, they are encapsulated into a gelatin-based capsule, using conventional capsule filling techniques. Each of the capsules includes 100 mg of Compound I, and is useful to be orally administered to a human or animal.

### Examples 15 and 16

A series of two compositions are prepared as follows.

Particles of Compound II, having a chemical structure as defined elsewhere herein, are provided which have diameters in excess of 100 microns. These particles are micronized in an Aljet mill (model 00 Jet-0-Mizer) to produce particles of Compound II having diameters in a range of 10-20 microns. Compound II has a solubility in water at 25° C. of less than 0.5 mg; and advancing/receding contact angles of greater than 90°/greater than 45°, respectively.

These small particles of Compound II are combined with particles of sodium dodecyl sulfate, a filler/diluent, such as microcrystalline cellulose, a disintegrant, such as pregelatinized starch, a glidant, such as fumed silica, and a lubricant, such as magnesium stearate to form two different compositions. The combined materials are mixed in blenders. The two mixtures have the following chemical make-ups.

CONCENTRATION, WT %

| | EXAMPLE | EXAMPLE |
|---|---|---|
| INGREDIENT | 15 | 16 |
| Compound II | 25 | 25 |
| Sodium Dodecyl Sulfate | 1.5 | 1.0 |
| Microcrystalline Cellulose | 64.5 | 65 |
| Pregelatinized Starch | 7.5 | 7.5 |
| Fumed Silica | 1.0 | 1.0 |
| Magnesium Stearate | 0.5 | 0.5 |

### Examples 17 and 18

One portion of each of Compositions 15 and 16 is pressed into a tablet, using a conventional tablet press. Each of the tablets includes 100 mg of Compound II, and is useful to be orally administered to a human or animal.

### Examples 19 and 20

Another portion of each of Compositions 15 and 16 is encapsulated into a gelatin-based capsule, using conventional capsule filling techniques. Each of the capsules includes 100 mg of Compound II, and is useful to be orally administered to a human or animal.

### Examples 21 and 22

A further portion of each of Compositions 15 and 16 is combined with a sufficient amount of water and mixed to form an extrudable mixture. Using conventional extrusion, cutting and drying equipment and techniques, each of the extrudable mixtures is extruded into thin strands, which are cut to form beads of suitable size to be placed into a capsule, and dried. After the beads are dried, they are encapsulated into a gelatin-based capsule, using conventional capsule filling techniques. Each of the capsules includes 100 mg of Compound II, and is useful to be orally administered to a human or animal.

### Examples 23 to 25

A series of three compositions are prepared as follows.

Particles of Compound III, having a chemical structure as defined elsewhere herein, are provided which have diameters in excess of 100 microns. These particles are micronized in an Aljet mill (model 00 Jet-0-Mizer) to produce particles of Compound III having diameters in a range of 10-20 microns.

These small particles of Compound III are combined with particles of sodium dodecyl sulfate, a filler/diluent, such as microcrystalline cellulose, a disintegrant, such as pregelatinized starch, a glidant, such as fumed silica, and a lubricant, such as magnesium stearate to form two different compositions. The combined materials are mixed in blenders. The two mixtures have the following chemical make-ups.

| | CONCENTRATION, WT % | | |
|---|---|---|---|
| | EXAMPLE | EXAMPLE | EXAMPLE |
| INGREDIENT | 23 | 24 | 25 |
| Compound III | 5 | 50 | 25 |
| Sodium Dodecyl Sulfate | 1.0 | 3.0 | 2.0 |
| Microcrystalline Cellulose | 85 | 38 | 64 |
| Pregelatinized Starch | 7.5 | 7.5 | 7.5 |
| Fumed Silica | 1.0 | 1.0 | 1.0 |
| Magnesium Stearate | 0.5 | 0.5 | 0.5 |

### Examples 26 to 28

One portion of each of Compositions 23, 24 and 25 is pressed into a tablet, using a conventional tablet press. The tablets of Compositions 23, 24 and 25 include 20 mg, 200 mg and 100 mg of Compound III, respectively. Each of the tablets is useful to be orally administered to a human or animal.

### Examples 29 to 31

Another portion of each of Compositions 23, 24 and 25 is encapsulated into a gelatin-based capsule, using conventional capsule filling techniques. The capsules of Compositions 23, 24 and 25 include 20 mg, 200 mg and 100 mg of Compound III, respectively. Each of the capsules is useful to be orally administered to a human or animal.

### Examples 32 to 34

A further portion of each of Compositions 23, 24 and 25 is combined with a sufficient amount of water and mixed to form an extrudable mixture. Using conventional extrusion, cutting and drying equipment and techniques, each of the extrudable mixtures is extruded into thin strands, which are cut to form beads of suitable size to be placed into a capsule, and dried. After the beads are dried, they are encapsulated into a gelatin-based capsule, using conventional capsule filling techniques. The capsules of Compositions 23, 24 and 25 include 20 mg, 200 mg and 100 mg of Compound III, respectively. Each of the capsules is useful to be orally administered to a human or animal.

### Examples 35 to 37

A series of three compositions are prepared as follows.

Particles of Compound IV, having a chemical structure as defined elsewhere herein, are provided which have diameters in excess of 100 microns. These particles are micronized in an Aljet mill (model 00 Jet-0-Mizer) to produce particles of Compound IV having diameters in a range of 10-20 microns. Compound IV has a solubility in water at 25° C. of less than 0.5 mg; and advancing/receding contact angles of greater than 90°/greater than 45°, respectively.

These small particles of Compound IV are combined with particles of sodium dodecyl sulfate, a filler/diluent, such as microcrystalline cellulose, a disintegrant, such as pregelatinized starch, a glidant, such as fumed silica, and a lubricant, such as magnesium stearate to form two different compositions. The combined materials are mixed in blenders. The two mixtures have the following chemical make-ups.

| | CONCENTRATION, WT % | | |
|---|---|---|---|
| | EXAMPLE | EXAMPLE | EXAMPLE |
| INGREDIENT | 35 | 36 | 37 |
| Compound IV | 2 | 20 | 50 |
| Sodium Dodecyl Sulfate | 0.5 | 1.0 | 3.0 |
| Microcrystalline Cellulose | 88.5 | 70 | 38 |
| Pregelatinized Starch | 7.5 | 7.5 | 7.5 |
| Fumed Silica | 1.0 | 1.0 | 1.0 |
| Magnesium Stearate | 0.5 | 0.5 | 0.5 |

### Examples 38 to 40

One portion of each of Compositions 35, 36 and 37 is pressed into a tablet, using a conventional tablet press. The tablets of Compositions 35, 36 and 37 include 10 mg, 100 mg and 250 mg of Compound IV, respectively. Each of the tablets is useful to be orally administered to a human or animal.

### Examples 41 to 43

Another portion of each of Compositions 35, 36 and 37 is encapsulated into a gelatin-based capsule, using conventional capsule filling techniques. The capsules of Compositions 35, 36 and 37 include 10 mg, 100 mg and 250 mg of Compound IV, respectively. Each of the capsules is useful to be orally administered to a human or animal.

### Examples 44 to 46

A further portion of each of Compositions 35, 36 and 37 is combined with a sufficient amount of water and mixed to form an extrudable mixture. Using conventional extrusion, cutting and drying equipment and techniques, each of the extrudable mixtures is extruded into thin strands, which are cut to form beads of suitable size to be placed into a capsule, and dried. After the beads are dried, they are encapsulated into a gelatin-based capsule, using conventional capsule filling techniques. The capsules of Compositions 35, 36 and 37 include 10 mg, 100 mg and 250 mg of Compound IV, respectively. Each of the capsules is useful to be orally administered to a human or animal.

While this invention has been described with respect to various specific examples and embodiments, it is to be understood that the invention is not limited thereto and that it can be variously practiced within the scope of the following claims.

1. A composition for administration to a gastro-intestinal tract of a human or animal, the composition comprising:
a therapeutic component consisting of an alpha-2-adrenergic receptor agonist and mixtures thereof wherein the therapeutic component is present in a physiologically effective amount that when absorbed into the gastro-intestinal tract of a human or animal the therapeutic component has the desired therapeutic effect on the human or animal, the therapeutic component having limited water solubility and limited surface wettability in water and being present as a plurality of solid particles;
a surfactant component in an amount effective to increase the absorption of the therapeutic component in a gastro-intestinal tract of a human or animal relative to a substantially identical composition without the surfactant component; and
a pharmaceutically acceptable carrier.

2-68. (canceled)

69. The composition of claim 1 wherein the therapeutic component has a water solubility at 25° C. of less than about 0.5 mg/ml.

70. The composition of claim 1 wherein the therapeutic component has sufficiently limited surface wettability in water so that solid particles of the therapeutic component are dissolved in water at a reduced rate relative to larger solid particles of the therapeutic component.

71. The composition of claim 1 wherein the plurality of particles have diameters in a range from the group consisting of 1 micron to about 50 microns, 1 micron to about 30 microns, and 3 microns to about 20 microns.

72. The composition of claim 1 wherein the therapeutic component has an advancing contact angle of about 90° or greater.

73. The composition of claim 1 wherein the therapeutic component has a receding contact angle of about 45° or greater.

74. The composition of claim 1 wherein the alpha-2-adrenergic receptor agonist component is selected from the group consisting of: 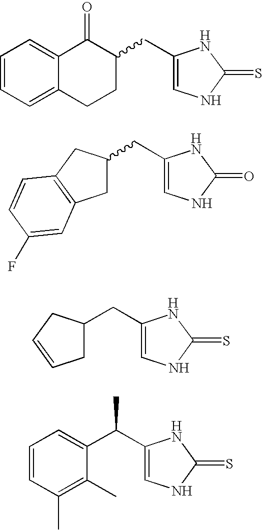
, pharmaceutically acceptable salts, esters, amides, stereoisomers and racemic mixtures thereof, and mixtures thereof.

75. The composition of claim 1 wherein the therapeutic component is 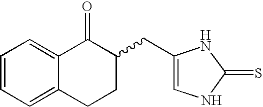
, pharmaceutically acceptable salts, esters, amides, stereoisomers, and racemic mixtures thereof, and mixtures thereof.

76. The composition of claim 1 wherein the therapeutic component is 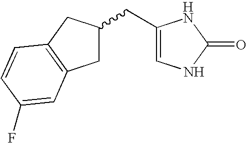
, pharmaceutically acceptable salts, esters, amides, stereoisomers, and racemic mixtures thereof, and mixtures thereof.

77. The composition of claim 1 wherein the therapeutic component is 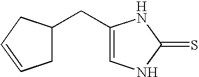
, pharmaceutically acceptable salts, esters, amides, stereoisomers, and racemic mixtures thereof, and mixtures thereof.

78. The composition of claim 1 wherein the therapeutic component is 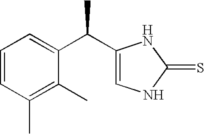
, pharmaceutically acceptable salts, esters, amides, stereoisomers, and racemic mixtures thereof, and mixtures thereof.

79. The composition of claim 1 wherein the surfactant component is effective to increase the surface wettability of the therapeutic component in water.

80. The composition of claim 1 wherein the surfactant component is selected from the group consisting of alkali metal hydrocarbyl sulfates, fatty acid ethoxylates, polyoxyethylene stearates, polyoxyethylene/polyoxypropylene copolymers and mixtures thereof.

81. The composition of claim 1 wherein the surfactant component is selected from the group consisting of alkali metal hydrocarbyl sulfates having about 8 to about 16 carbon atoms per molecule and mixtures thereof, sodium hydrocarbyl sulfates having about 8 to about 16 carbon atoms per molecule and mixtures thereof, and sodium dodecyl sulfate.

82. The composition of claim 1 wherein the desired therapeutic effect includes treatment of the following disorders: chronic pain; neurological conditions; sensory hypersensitivity such as that associated with fibromyalgia or headaches such as migraines; gastrointestinal diseases such as irritable bowel syndrome and dyspepsia; dermatological conditions such as psoriasis; cardiovascular disorders; tachycardias; disorders of peripheral vasoconstriction including Raynaud's Syndrome and scleroderma; panic attacks; metabolic disorders such as type II diabetes, insulin-resistance and obesity; disorders of muscle contraction including disorders of skeletal muscle contraction, disorders of smooth muscle contraction, spasticity, and disorders of muscle contraction associated with tension-type headache; behavioral disorders such as, but not limited to, over-eating and drug dependence; and sexual dysfunction.
